(19) United States
(12) Patent Application Publication
(10) Pub. No.: US 2017/0156631 A1
(43) Pub. Date: Jun. 8, 2017

(54) DEVICE FOR AN ELECTRICAL IMPEDANCE TOMOGRAPH

(71) Applicant: Drägerwerk AG & Co. KGaA, Lübeck (DE)

(72) Inventors: Frank SATTLER, Lübeck (DE); Karsten HILTAWSKY, Stockelsdorf (DE); Jian-Hua LI, Lübeck (DE)

(21) Appl. No.: 15/323,546

(22) PCT Filed: Jul. 1, 2015
(86) PCT No.: PCT/EP2015/001321

§ 371 Date: Jan. 3, 2017

(30) Foreign Application Priority Data
Jul. 4, 2014 (DE) 10 2014 009 889.3

(51) Int. Cl. A61B 5/053; H01R 13/66

(52) U.S. Cl. CPC A61B 5/0536; H01R 13/6666; A61B 2562/182; A61B 2562/0214; A61B 2562/043; A61B 2562/222; A61B 2562/227; H01R 2201/12

(57) ABSTRACT

A connection unit (1) connects an impedance tomograph (3) electrically to first and second skin electrodes (6, 8). The skin electrodes (6, 8) are arranged on an electrode carrier (4) spaced from one another in a longitudinal direction L. First and second opposite contact devices (24, 26) are arranged on the electrode carrier (4) and are respectively connected to associated electrodes (6, 8). First and second contact devices (16, 18) are configured for respective detachable connection to the associated first and second opposite contact devices (24, 26). Each contact device (16, 18) has an electric protective circuit (10, 12). A system is provided for an electrical impedance tomograph (3), which system has the connection unit (1).

### CROSS REFERENCE TO RELATED APPLICATIONS

This application is a United States National Phase Application of International Application PCT/EP2015/001321 filed Jul. 1, 2015, and claims the benefit of priority under 35 U.S.C. §119 of German Application 10 2014 009 889.3 filed Jul. 4, 2014, the entire contents of which are incorporated herein by reference.

### FIELD OF THE INVENTION

The present invention pertains to a connection unit, by means of which an impedance tomograph can be electrically connected to a first skin electrode and to a second skin electrode of an electrode carrier, wherein the skin electrodes are arranged at mutually spaced locations from one another on the electrode carrier in the longitudinal direction of the electrode carrier; a first opposite contact device is arranged on the electrode carrier and is connected to the first skin electrode; a second opposite contact device is arranged at the electrode carrier and is connected to the second skin electrode; the connection unit has a first contact device and a second contact device; the first contact device is configured such that it can be detachably connected to the first opposite contact device, and the second contact device is configured such that it can be detachably connected to the second opposite contact device.

### BACKGROUND OF THE INVENTION

Electrodiagnostic methods are frequently employed on patients who are in a critical condition. It may become necessary here to use a defibrillator for a short time, without there being enough time for properly disconnecting diagnostic devices from the patient. This leads to the risk of damage to the diagnostic devices due to overvoltage pulses.

Defibrillation is the only effective and life-saving procedure in life-threatening situations such as ventricular fibrillation or pulseless ventricular tachycardia.

Any delay that would arise from the removal of electrodes or electric terminals from the patient is to be avoided.

Input resistances in the range or 10 kohms to 50 kohms are used according to the state of the art in pure electrocardiogram (ECG or EKG) devices or in combined ECG/impedance-measuring devices that are not used for imaging methods in order to prevent technical damage due to the use of defibrillators.

The special difficulty in impedance-tomographic methods is that, unlike in pure electrocardiography, the electrodes are often provided for a dual purpose in applications in thoracic electrical impedance tomography.

First, they shall introduce into the patient the stimulating currents, which may reach up to 10 mA and with which a readily analyzable potential distribution shall be achieved in the patient.

Second, they shall feed the low signal voltages, which are measured on the skin surface of the patient based on the potential distribution generated with the stimulating currents, to the input amplifier or to an analysis unit. The signal voltages to be measured may be in the μV to mV range.

The currents to be introduced must be selected at high values, up to 10 mA, in order to generate sufficient potential differences in the entire thorax in order to be able to generate an image from the potentials picked off (sensed/detected). Voltages of 100 V to 500 V would drop over resistances of 10 kohms to 50 kohms. Such voltages cannot be used on the patient, and the possibility of protecting the impedance tomograph by sufficiently high protective resistors cannot be taken into consideration.

Protecting the inputs by varistors or diodes connected in parallel to the input amplifier is likewise problematic. Additional stray capacitances, which are connected between the signal line and the reference potential, must be kept as low as possible. This is necessary to prevent unacceptable reactive impedances, which are located parallel to the input of the first amplifier stage, from developing at the usual operating frequencies of about 10 kHz to 200 kHz. They would unacceptably increase the load, which the measuring circuit represents against the potential distribution on the skin surface of the patient and thus they would distort the measurement. At a frequency of 50 kHz, 10 pF already represents an impedance of about 30 kohms. Thus, solutions that contain varistors or diodes connected in parallel to the input amplifier are disadvantageous if the parasitic capacitance of these varistors or diodes is higher than a few pF. However, this rules out all types that could dissipate the currents that usually develop due to a defibrillator shock.

It must therefore be assumed that the defibrillator is used without the patient being disconnected from the impedance tomograph. In addition to the necessary protection of the impedance tomograph from an overload, it becomes necessary to avoid an excessive draining of the energy of the defibrillator pulse in order not to unacceptably limit the efficiency of the defibrillator.

For example, standards require that a maximum of 10% of the energy of a defibrillator pulse may be dissipated by the measuring circuit if the effectiveness shall be assumed to be still sufficient.

Equivalent standard specifications for thoracic impedance tomographs can undoubtedly be expected in case this diagnostic method becomes established. While components of the device that are possibly at risk must be protected, effective use of the defibrillator for the protection of the patient must be guaranteed.

Therefore, there was a requirement to provide a device that offers reliable protection against damage caused by overvoltage for an electrical impedance tomograph connected to a patient in case of application of a defibrillator, without the risk of draining of the defibrillator pulses to an extent that is disadvantageous for the effectiveness of defibrillation, and the impedance tomograph shall continue to be able to function at the same time.

A device for protecting an electrical impedance tomograph, which meets the above-mentioned requirement, is known, for example, from the document DE 10 2005 041 385 A1. DE 10 2005 041 385 A1 discloses an electrical impedance tomograph, whose signal inputs are provided with a protective circuit, which protects the signal inputs against excessively high input currents when a voltage that is excessively high for the normal measuring operation, the protective circuit being integrated in an electrode carrier used with the impedance tomograph.

Electrode carriers with a plurality of skin electrodes, which are arranged on the electrode carrier at mutually spaced locations from one another in a longitudinal direction of the electrode carrier, are usually used in practice. For example, at least two, three, five, ten, fifteen or more skin electrodes may thus be provided. If the device used in the above-mentioned document is now used to protect the electrical impedance tomograph, all skin electrodes are first to be connected electrically to the protective circuit in order to reliably protect the electrical impedance tomograph arranged downstream in the signal conduction direction against damages caused by overvoltage. The electrical connections between the protective circuit preferably integrated in the electrode carrier and the plurality of skin electrodes are each shielded specially electrically in order to prevent the user from being inadvertently exposed to the above-mentioned high voltages of about 100 V to 500 V. In other words, the skin electrodes are to be connected by specially configured electrical line connections to the one protective circuit integrated in the electrode carrier in a Y-shaped manner.

It was found in the case of the aforementioned Y-shaped type of connection that corresponding electrical lines can often be manufactured in a very complicated manner and are therefore very expensive, because, in addition to the protection to be provided, such connections also must offer high flexibility in order for the electrode carrier to be able to be placed on the patient's body without a gap to the extent possible.

### SUMMARY OF THE INVENTION

A basic object of the present invention is therefore to provide a cost-effective overvoltage protection, which can be manufactured in a simple manner, for a system comprising impedance tomographs and a flexible electrode carrier and a plurality of skin electrodes, wherein the overvoltage protection shall reliably protect the impedance tomograph against damage caused by overvoltage in case of the use of a defibrillator. In particular, the above-mentioned problems shall be eliminated.

According to a first aspect of the present invention, the object is accomplished by a connection unit, by means of which an impedance tomograph can be electrically connected to a first skin electrode and to a second skin electrode of an electrode carrier. The skin electrodes are arranged on the electrode carrier at mutually spaced locations from one another in the longitudinal direction. A first opposite contact device is arranged on the electrode carrier and is connected to the first skin electrode. A second opposite contact device is arranged on the electrode carrier and is connected to the second skin electrode. The connection unit of the present invention has a first contact device and a second contact device. The first contact device is configured such that the first contact device is detachably connectable to the first opposite contact device. The second contact device is configured such that the second contact device is detachably connectable to the second opposite contact device. The first contact device and the second contact device have a respective electric protective circuit each. The protective circuits are preferably overvoltage protection circuits. The protective circuits may be configured, in principle, to block and/or to damp predefined signals, especially beginning from a predefined increase in amplitude, beginning from a certain frequency, beginning from a certain voltage and/or beginning from a certain current.

The present invention is based on the discovery that a central protective circuit occupies a comparatively large space, so that if such a protective circuit is integrated in the electrode carrier, the latter will lose its necessary flexibility to be able to be placed on the body of a patient without gaps. In addition, it was determined that the electrical connection lines are preferably configured in a Y shape in relation to the skin electrodes. These connection lines should be both flexibly deformable and configured as electrically shielding and protected lines. Such electrical connection lines can be manufactured at a very high cost only. The basic idea of the present invention is therefore to provide a plurality of protective circuits, especially two protective circuits, which can be connected each to different skin electrodes. The skin electrodes are arranged on the electrode carrier. In addition, opposite contact elements are provided for the electrode carrier for the electrical contacting in order to electrically connect the skin electrodes to the impedance tomograph. Provisions are now made according to the present invention for at least the opposite contact elements, which are electrically connected to the first skin electrode or to the second skin electrode, to be able to be connected to a respective contact device according to the present invention each, the contact devices having a respective protective circuit each. A protective circuit is preferably meant to be an overvoltage protective circuit. It can thus be guaranteed that the protective circuits can be arranged close to the electrodes, The electrical connections between the protective circuits and the respective, at least one skin electrode that can be coupled can be configured as very short connections, since the corresponding connection extends at least essentially between the coupled skin electrode and the connected opposite contact element. The connection is consequently very short and can therefore be manufactured in a cost-effective manner. In addition, the protective circuits according to the present invention are electrically coupled with a respective skin electrode each or are electrically coupled with a small number of skin electrodes only, for example, with a maximum of two, three or four skin electrodes. The protective circuits can therefore be configured each as very compact protective circuits. The opposite contact elements are arranged on the electrode carrier on the outer side for electrical contacting. If the contact devices are electrically connected each to one of the opposite contact elements, the contact devices can also be fastened to the respective opposite contact element. Therefore, each of the contact devices preferably has a terminal, which can be mechanically connected to the opposite contact device. A plug-type connection is especially preferably formed between each of the contact devices and the corresponding opposite contact device. As an alternative, fastening to the electrode carrier is possible. However, due to their compact type of construction, the contact devices do not hinder the flexibility of the electrode carrier, since, analogously to the skin electrodes, the opposite contact elements are located at mutually spaced locations from one another in the longitudinal direction.

An advantageous embodiment of the connection unit is characterized by an electrical line connection, at one, especially multipart end of which the first contact device and the second contact device are arranged, and whose other end is configured such that it can be connected to the impedance tomograph. Corresponding terminals may be provided at the end of the line connection that can be connected to the impedance tomograph. The other end of the line connection may be a multipart end. This means that the corresponding end section of the line connection is split into a plurality of electrical lines. The contact devices are then arranged at the end of these lines. The lines preferably converge from their respective end at the contact devices in the aforementioned end section of the line connection in a Y-shaped manner. As was explained above, the contact devices are used to connect the skin electrodes or the respective corresponding opposite contact devices to the electrical line connection and to the impedance tomograph. The number of line connections for connecting skin electrodes to an impedance tomograph can be minimized with a line connection, one end of which has a multipart configuration for arranging the contact devices. Only one line connection is preferably necessary between the impedance tomograph and the contact devices or skin electrodes.

Another advantageous embodiment of the connection unit is characterized in that the electrical line connection and the contact devices form one unit. The electrical line connection and the at least two contact devices thus form a common unit, wherein their respective functional sections adjoin each other. Their functions, i.e., the transmission of electrical signals in case of the line connection and the blocking and/or damping of certain types of signals in the protective circuit, continue to be separated. The establishment of the contact between the impedance tomograph and the skin electrode thus becomes simpler, since the aforementioned unit can be used by a person installing a corresponding structure in an especially simple manner and rapidly. In addition, the risk of incorrect installation of the line connection and of the contact devices decreases.

Another advantageous embodiment of the connection unit is characterized in that the protective circuits have a capacitor and/or passive components each. The protective circuits are electrical protective circuits. Consequently, the transmission of predefined electrical signals shall be prevented and/or modified, especially damped, in such a way, before the signals compromise devices and/or electrical units arranged downstream. Protective circuits with one capacitor each proved to be advantageous for preventing the transmission of high voltages. The protective circuits are therefore preferably overvoltage protective circuits. Additional passive components likewise proved to be advantageous, because such passive components are very robust. Passive components are therefore suitable for use with a long service life.

Another advantageous embodiment of the connection unit is characterized in that the protective circuits each have electrical shielding. With the electrical shielding, the intended mode of operation of the protective circuits is prevented from being affected by external electrical potentials. The reliability of operation of the protective circuits is thus guaranteed especially simply and effectively.

According to another aspect of the invention, the object is accomplished by a system. The system is a system for an electrical impedance tomograph with an electrode carrier; with a first skin electrode and with a second skin electrode, which are arranged at mutually spaced locations from one another on the electrode carrier in the longitudinal direction of the electrode carrier; with a first opposite contact device, which is arranged on the electrode carrier and is connected to the first skin electrode; and with a second opposite contact device, which is arranged on the electrode carrier and is connected to the second skin electrode; wherein the system has a connection unit according to the present invention; the first contact device is detachably connected to the first opposite contact device; and the second contact device is detachably connected to the second opposite contact device.

Features, details and advantages, which are described in connection with the connection unit according to the present invention, also apply, of course, in connection with the system according to the present invention and vice versa, so that reference is and can always mutually be made concerning the disclosure of the individual aspects of the present invention.

An advantageous embodiment of the system is characterized in that the impedance tomograph is associated with the system, wherein the impedance tomograph is electrically connected to the first skin electrode and to the second skin electrode by means of the electrical line connection. At least the first contact device and the second contact device are especially preferably associated with the electrical line connection. The line connection can thus be connected at one end to the impedance tomograph and to the opposite contact devices with the other end, at which the contact devices are arranged, in order to electrically connect the skin electrodes to the impedance tomograph. The impedance tomograph is protected with the electrical protective circuits, which are enclosed by the contact devices. In particular, the impedance tomograph is prevented from being exposed to an excessively high voltage, caused especially by a defibrillator.

Another advantageous embodiment of the system is characterized in that the protective circuits are each a part of a multipart protective circuit unit, wherein additional parts of the protective circuit unit are integrated in the electrode carrier, in the electrical line connection, in the skin electrodes and/or in the impedance tomograph. The protective circuits are defined as the protective circuits of the contact devices. The protective circuits are preferably configured here as overvoltage protective circuits. Certain voltages, which exceed a certain amplitude, are prevented with such an overvoltage protective circuit from being transmitted. Additional components may be provided if provisions are made for preventing the transmission of other types of signals as well. Only frequencies from a predefined frequency spectrum may be transmitted to the impedance tomograph in order to protect the impedance tomograph against signals with a frequency outside the predefined frequency spectrum. Thus, a corresponding filtering may be provided. This may form an additional part of the protective circuit unit. The filter structure may be integrated, for example, in the electrical line connection. However, the part of each protective circuit unit that protects against overvoltages shall preferably be comprised by the corresponding contact device.

Further advantageous features of the present invention will appear from the description of the embodiments according to the present invention together with the attached drawings. Embodiments according to the present invention may accomplish individual features or a combination of a plurality of features. The present invention will be described below without limitation of the general inventive idea on the basis of an exemplary embodiment with reference to the drawings. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic sectional view of the connection unit according to the present invention.

### DESCRIPTION OF THE PREFERRED EMBODIMENTS

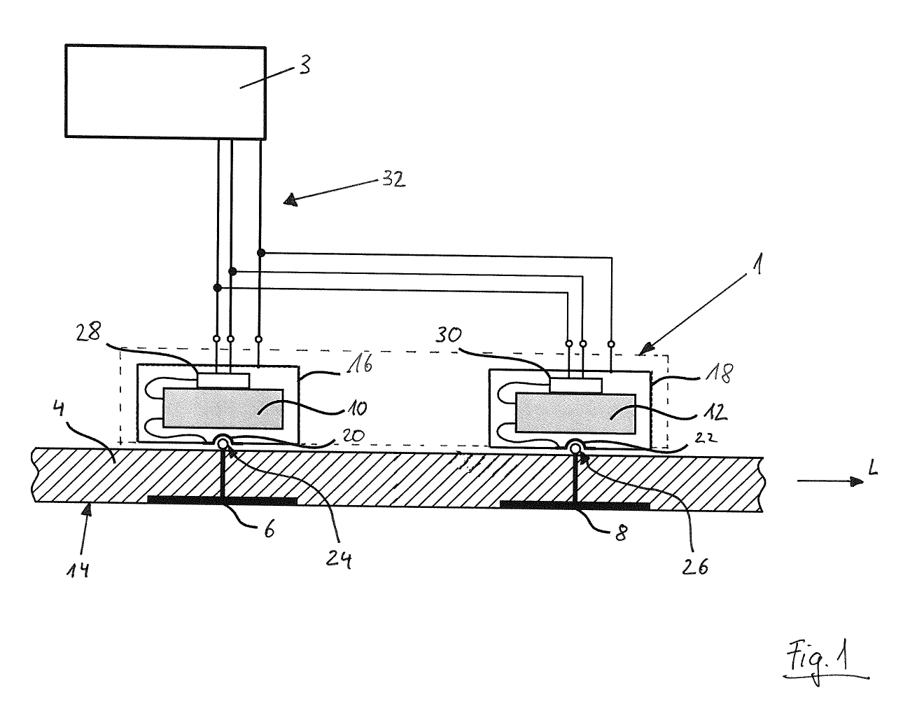

Referring to the drawings, FIG. 1 schematically shows the connection unit 1 according to the present invention (box in broken lines) for an electrical impedance tomograph 3. The connection unit 1 comprises a first contact device 16 and a second contact device 18. The contact devices 16, 18 may be configured as devices that are separate from one another. As an alternative, provisions may be made for the contact device to be mechanically connected or coupled with one another in 16, 18.

FIG. 1 shows, in addition, an electrode carrier 4. It is preferably a so-called electrode belt. The electrode carrier 4 may consequently be placed like a belt around the body of a patient. To transmit the electrical signals necessary for the impedance tomography to the body or to pick up electrical signals, the first and second skin electrodes 6, 8 are arranged on the electrode carrier 4 at mutually spaced locations from one another in the longitudinal direction L of the electrode carrier 4. If the electrode carrier 4 is bent in a ring-shaped manner, the skin electrodes 6, 8 are arranged radially on the inner side. The skin electrodes 6, 8 will consequently come directly into contact with the patient's body and they can thus guarantee the transmission of the desired signals. As can be seen in FIG. 1, the skin electrodes 6, 8 can end continuously flush with an end face 14 of the electrode carrier 4. Thus, they form a common surface of the electrode carrier 4 or of the device 2.

On the side facing away from the end face 14 of the electrode carrier 4, each of the skin electrodes 6, 8 are connected to a respective opposite contact device 24, 26. Thus, the first skin electrode 6 is connected to the first opposite contact device 24, which is arranged on the rear side of the electrode carrier 4. The second skin electrode 8 is connected to the second opposite contact device 26, which is likewise arranged on the rear side of the electrode carrier 4. The two opposite contact devices are configured each as a terminal element and protrude over the electrode carrier 4. The two opposite contact devices 24, 26 are located at spaced locations from one another in the longitudinal direction L of the electrode carrier 4. The contact devices 16, 18 can be connected to the opposite contact devices 24, 26 in an electrically and mechanically detachable manner. The contact devices 16, 18 may also be completely fastened mechanically to the opposite contact device 24, 26.

It may happen, in a situation that is critical for a patient, that the impedance tomograph 3 and a defibrillator are used simultaneously. High voltage pulses are emitted by the defibrillator. To prevent the voltage pulses emitted by the defibrillator from damaging or even destroying the impedance tomograph 3, provisions are made according to the present invention for the first contact device 16 to have an electrical protective circuit 10 and for the second contact device 18 to have an electrical protective circuit 12. A voltage pulse of the defibrillator, which is sent from a skin electrode 6, 8 to the corresponding contact device 16, 18, will then reach the corresponding protective circuit 10, 12, which prevents the high voltage pulse from being transmitted. The impedance tomograph 3 is consequently effectively protected against such voltage pulses.

An electrical line connection 32, i.e., preferably a bundle of electrical lines, is provided to establish an electrical connection between the contact devices 16, 18 and the impedance tomograph 3. The line connection 32 extends from a terminal of the impedance tomograph 3 to the first opposite contact device 24 and to the second opposite contact device 26. One end of the electrical line connection 32 therefore preferably has a multipart configuration. The electrical line connection 32 consequently has an end section, which is split into a plurality of electrical lines.

For a simple handling of the electrical line connection 32 and of the contact devices 16, 18, the electrical line connection and the contact devices 16, 18 form one unit. To connect the defibrillator 3 to the skin electrodes 6, 8, the aforementioned unit is connected with one end to the impedance tomograph 3 and with the other end, at which the first contact device 16 and the second contact device 18 are arranged, to the first opposite contact device 24 and to the second opposite contact device 26. A quick connection, especially a plug-type connection, is preferably formed between the first contact device 16 and the first opposite contact device 24. Thus, the opposite contact device 16 may have a first receptacle 20, into which the opposite contact device 24 can be plugged in order to subsequently establish the mechanical and/or electrical connection between the first contact device 16 and the first opposite contact device 24. The plug-type connection may also have a reversed configuration. Analogously to the first contact device 16, the second contact device 18 also has a second receptacle 22, into which the second opposite contact device 26 can be plugged in order to then establish the mechanical and/or electrical connection between the second contact device 18 and the second opposite contact device 26. A reversed plug-type connection is possible here as well.

FIG. 1 shows, in addition, that the first receptacle 20 of the first contact device 16 is electrically connected to the first protective circuit 10 and the first protective circuit 10 is, in turn, connected to a first preprocessing unit 28. The first preprocessing unit 28 is preferably associated with the first contact device 16. The first receptacle 20, the first protective circuit 10 and the first preprocessing unit 28 are consequently connected electrically in series and are preferably enclosed by the first contact device 16. The preprocessing unit 28 may be configured as a processing unit for electric signals, for example, as a data processing unit, as a measuring analysis unit or the like. As an alternative, the preprocessing unit 28 may be configured as a signal-amplifying unit. The preprocessing unit 28 has active electrical components for both alternatives. Based on the above-mentioned series connection, the first protective circuit 10 already protects the active components of the first contact device 16. The first preprocessing unit 28 is joined by the electrical line connection 32 in order to connect the first contact device 16 to the impedance tomograph 3. Just like the first preprocessing unit 28 as well, the impedance tomograph 3 with its active electrical components is also protected by the first protective circuit 10.

The second contact device 18 is designed and/or configured analogously to the first contact device 16. Reference is therefore analogously made to the above explanations for the second contact device 18. Thus, the second contact device 18 has, for example, a series connection of a second receptacle 22, of the second protective circuit 12 and of a second preprocessing unit 30. The second preprocessing unit 30 is joined by the electrical line connection 32 to the impedance tomograph 3. Therefore, the analogous effects and/or advantages apply.

While specific embodiments of the invention have been shown and described in detail to illustrate the application of the principles of the invention, it will be understood that the invention may be embodied otherwise without departing from such principles.

1. A connection unit with which an impedance tomograph can be electrically connected to a first skin electrode and a second skin electrode of an electrode carrier, wherein the skin electrodes are arranged on the electrode carrier at mutually spaced locations from one another in a carrier longitudinal direction, a first opposite contact device is arranged on the electrode carrier and is connected to the first skin electrode, a second opposite contact device is arranged on the electrode carrier and is connected to the second skin electrode, the connection unit comprising:
a first contact device configured to be detachably connected to the first opposite contact device; and
a second contact device configured to be detachably connected to the second opposite contact device, wherein the first and second contact devices each have a respective electric protective circuit.

2. A connection unit in accordance with claim 1 further comprising an electrical line connection comprising a contact device end with connections to the first contact device and the second contact device and an impedance tomograph end configured to be connected to the impedance tomograph.

3. A connection unit in accordance with claim 2, wherein the electrical line connection and the contact devices form one unit.

4. A connection unit in accordance with claim 1, wherein the protective circuits each comprise a capacitor or passive components or both a capacitor and passive components.

5. A connection unit in accordance with claim 1, wherein the protective circuits each comprise electrical shielding.

6. A system for an electrical impedance tomograph, the system comprising:
an electrode carrier comprising:
a first skin electrode and a second skin electrode, which are arranged on the electrode carrier at mutually spaced locations from one another in a longitudinal direction of the electrode carrier
a first opposite contact device arranged on the electrode carrier and connected to the first skin electrodes; and
a second opposite contact device arranged on the electrode carrier and connected to the second skin electrode; and
a connection unit comprising:
a first contact device configured to be detachably connected to the first opposite contact device; and
a second contact device configured to be detachably connected to the second opposite contact device, wherein the first and second contact devices each have a respective electric protective circuit.

7. A system in accordance with claim 6, wherein:
the connection unit further comprises an electrical line connection comprising a contact device end with connections to the first contact device and the second contact device and an impedance tomograph end configured to be connected to the impedance tomograph; and
the impedance tomograph is associated with the system and the impedance tomograph is electrically connected to the first and second skin electrodes by the electrical line connection.

8. A system in accordance with claim 7, wherein:
the protective circuits are each a part of a multipart protective circuit unit; and
additional parts of the protective circuit unit are integrated in the electrical line connection or are integrated in the skin electrodes or are integrated in the impedance tomograph or are integrated in any combination of the electrical line connection and the skin electrodes and the impedance tomograph.

9. A system in accordance with claim 7, wherein the electrical line connection and the contact devices form one unit.

10. A system in accordance with claim 6, wherein the protective circuits each comprise a capacitor or passive components or both a capacitor and passive components.

11. A system in accordance with claim 6, wherein the protective circuits each comprise electrical shielding.
